(19) United States
(12) Patent Application Publication
(10) Pub. No.: US 2024/0350521 A1
(43) Pub. Date: Oct. 24, 2024

(54) COMPOSITIONS AND METHODS FOR PREVENTING OR TREATING PRECOCIOUS PUBERTY

(71) Applicant: TAIPEI MEDICAL UNIVERSITY, Taipei City (TW)

(72) Inventor: Yang-Ching CHEN, Taipei City (TW)

(73) Assignee: TAIPEI MEDICAL UNIVERSITY, Taipei City (TW)

(21) Appl. No.: 18/641,154

(22) Filed: Apr. 19, 2024

Related U.S. Application Data
(60) Provisional application No. 63/497,515, filed on Apr. 21, 2023.

(51) Int. Cl. A61K 31/704; A61P 5/24; A61P 15/00

(52) U.S. Cl. CPC A61K 31/704; A61P 5/24; A61P 15/00

(57) ABSTRACT

Provided is a method for preventing or treating precocious puberty in a subject in need thereof. The method includes administering to the subject an effective amount of glycyrrhizin or a salt or derivative thereof and an acceptable excipient thereof. Also provided is a composition for use in preventing or treating precocious puberty in a subject in need thereof.

### CROSS-REFERENCE TO RELATED APPLICATION

This non-provisional application claims the benefit under 35 U.S.C. § 119(e) to U.S. Provisional Application No. 63/497,515, filed Apr. 21, 2023, all of which are hereby expressly incorporated by reference into the present application.

### REFERENCE TO ELECTRONIC SEQUENCE LISTING

The application contains a Sequence Listing which has been submitted electronically in .XML format and is hereby incorporated by reference in its entirety. Said .XML copy, created on Apr. 12, 2024, is named “TPMU-0009US_Sequence listing-20240412.xml” and is 4,096 bytes in size. The sequence listing contained in this .XML file is part of the specification and is hereby incorporated by reference herein in its entirety.

### TECHNICAL FIELD

The present disclosure relates to a method for preventing or treating precocious puberty, and particularly to a method for preventing or treating precocious puberty by administering to a subject in need thereof a composition comprising glycyrrhizin or a salt or derivative thereof and an acceptable excipient thereof. Also related is a composition for use in preventing or treating precocious puberty in a subject in need thereof.

### BACKGROUND

Precocious puberty (PP) is characterized by the early development of secondary sexual characteristics to a child of at least 2 years of age, which may lead to many adverse outcomes. PP is further divided into two classifications: peripheral precocious puberty (PPP) or central precocious puberty (CPP). PPP is defined by early sexual development prompted by sex steroids resulting from abnormal endogenous or exogenous sources such as disease or environmental exposure. PPP-associated symptoms, such as ambiguous genital development or virilization in females, can result from insufficient androgen levels caused by various tumors (i.e., gonadal, adrenal, and germ cell tumors). On the other hand, CPP is defined by early sexual development prompted by production and release of gonadotropins and/or sex steroids from normal endogenous sources including the hypothalamus or pituitary.

Gonadotropin-releasing hormone analog (GnRHa) is currently the first-line treatment for CPP. Sustained delivery of GnRH or GnRH agonists results in chronic stimulation of GnRH receptors in the pituitary, which leads to subsequent downregulation of GnRH receptor activity after an initial transient increase. Downregulation of GnRH receptor activity reduces GnRH-dependent secretion of gonadotropins, including luteinizing hormone (LH) and follicle-stimulating hormone (FSH), which are key drivers of normal development during puberty.

However, GnRHa is a costly and invasive medication that may create an unpleasant experience for patients, especially for children as young as 2 years of age, who need the treatment for years. Therefore, there is an unmet need for an effective and tolerable treatment that may balance patient compliance and comfort, so as to effectively prevent, alleviate, or treat precocious puberty and be helpful in height growth.

### SUMMARY

The present disclosure provides a composition and a method for preventing or treating precocious puberty, e.g., central precocious puberty and peripheral precocious puberty. The composition of the present discourse comprises glycyrrhizin or a salt or derivative thereof and a pharmaceutically acceptable excipient thereof or a food excipient thereof. When administered, the composition is capable of inhibiting estrogen production in a subject, thereby representing an alternative strategy for the prevention, alleviation, or treatment of precocious puberty. Also, the composition of the present discourse is safe for the human body.

In at least one embodiment of the present disclosure, a composition for preventing or treating precocious puberty is provided. The composition comprises glycyrrhizin or a salt or derivative thereof and at least one of a pharmaceutically acceptable excipient thereof and a food excipient thereof.

In at least one embodiment of the present disclosure, the salt or derivative of the glycyrrhizin is selected from the group consisting of glycyrrhizic acid, glycyrrhetic acid, monoammonium glycyrrhizinate, dipotassium glycyrrhizinate, sodium glycyrrhizinate, and calcium glycyrrhizinate.

In at least one embodiment of the present disclosure, the glycyrrhizin or the salt or derivative thereof serves as a sole active ingredient for preventing or treating the precocious puberty in the composition. In some embodiments, the glycyrrhizin or the salt or derivative thereof is in combination with an additional active ingredient for preventing or treating the precocious puberty.

In at least one embodiment of the present disclosure, the composition provided herein may be a pharmaceutical composition or an edible composition. In some embodiments, the pharmaceutical composition of the present disclosure comprises glycyrrhizin or a salt or derivative thereof and a pharmaceutically acceptable excipient thereof. In some embodiments, the edible composition of the present disclosure comprises glycyrrhizin or a salt or derivative thereof and a food excipient thereof.

In at least one embodiment of the present disclosure, the glycyrrhizin or the salt or derivative thereof is present in an amount of from about 0.001% to about 95% of the composition by weight, such as 0.001%, 0.002%, 0.005%, 0.01%, 0.02%, 0.05%, 0.08%, 0.1%, 0.2%, 0.3%, 0.4%, 0.5%, 0.75%, 1%, 2%, 5%, 10%, 15%, 20%, 25%, 30%, 35%, 40%, 45%, 50%, 55%, 60%, 70%, 80%, 90%, and 95%. In some embodiments, an amount of the glycyrrhizin or the salt or derivative thereof in the composition has a lower limit chosen from 0.001%, 0.005%, 0.010%, 0.05%, 0.1%, 0.5%, 1%, 5%, 10%, 15%, and 20% by weight, and an upper limit chosen from 95%, 90%, 80%, 70%, 60%, 50%, 40%, 30%, and 25% by weight. In some embodiments, the glycyrrhizin or the salt or derivative thereof is in an amount of from about 0.01% to about 80% by weight in the composition.

In at least one embodiment of the present disclosure, a method for preventing or treating precocious puberty in a subject in need thereof is also provided. The method comprises administering an effective amount of the above-mentioned composition to the subject.

In at least one embodiment of the present disclosure, the glycyrrhizin or the salt or derivative thereof is administered to the subject in an effective amount of from about 0.1 mg/day to about 2,000 mg/day. For example, the glycyrrhizin or the salt or derivative thereof may be administered to the subject in an effective amount of from about 0.5 mg/day to about 1,000 mg/day, from about 1 mg/day to about 800 mg/day, from about 5 mg/day to about 500 mg/day, and from about 10 mg/day to about 300 mg/day.

In at least one embodiment of the present disclosure, after the administration, the composition may inhibit early ovary growth, inhibit estrogen production, inhibit short chain fatty acids production, and/or increase body length in the subject.

In at least one embodiment of the present disclosure, after the administration, the composition may change gut microbiome in the subject. For example, the composition may increase a level of at least one of Dialister, Atopobium, Granulicatella, family Carnobacteriaceae, Romboutsia, and Allobaculum in the subject, and may decrease a level of at least one of Lachnospirace, Prevotellaceae, Coprococcus, and Ruminococcaceae in the subject.

In at least one embodiment of the present disclosure, the subject may be a child suffering or suspected of suffering from the precocious puberty. In some embodiments, the subject may be of age from 2 to 18 years old, such as from 5 to 12 years old, from 6 to 16 years old, from 8 to 15 years old, and from 9 to 17 years old.

In at least one embodiment of the present disclosure, the composition may be administered to the subject in a period ranging from 1 month to 10 years, such as from 2 months to 8 years, from 3 months to 5 years, and from 6 months to 3 years.

### BRIEF DESCRIPTION OF THE DRAWINGS

The present disclosure can be more fully understood by reading the following detailed descriptions of the embodiments, with reference made to the accompanying drawings.

FIG. 1 shows the principal coordinate analysis plot based on Bray-Curtis dissimilarity.

FIG. 2 shows the analysis results of linear discriminant analysis effect size (LEfSE) in each group.

FIG. 3 shows the results of survival analyses of puberty-related timepoints.

FIG. 4 shows the estrogen levels (pg/mL) of rats in each group on sacrifice day.

FIG. 5 shows the alpha diversity of rats referencing to the danazol group.

FIG. 6 shows the principal coordinate analysis plot of rats in each group based on Bray-Curtis dissimilarity.

FIG. 7 shows the operational taxonomic units (OTUs) at the phylum level of rats in each group.

FIG. 8 shows the LEfSE analysis of rats in each group. The taxa from top to bottom refer to 1: g_Romboutsia; 2: g_Parabacteroides; 3: f_Tannerellaceae; 4: p_Verrucomicrobia; 5: o_Verrucomicrobiales; 6: f_Akkermansiaceae; 7: g_Akkermansia; 8: c_Verrucomicrobiae; 9: g_Lachnoclostridium; 10: g_Muribaculum; 11: g_Ruminococcaceae_UBA1819; 12: g_Clostridium_innocuum; 13: g_Fusicatenibacter; 14: g_Streptococcus; 15: f_Streptococcaceae; 16: g_Ruminococcaceae_UCG_008; 17: g_Shuttleworthia; 18: g_Erysipelotrichaceae_Uncultured; 19: g_Allobaculum; 20: g_ClostridialesvadinBB60_Uncultured; 21: f_Clostridiales_vadinBB60; 22: g_Ruminiclostridium_9; 23: g_Anaerostipes; 24: c_Gammaproteobacteria; 25: p_Proteobacteria; 26: g_Escherichia_Shigella; 27: f_Enterobacteriaceae; 28: o_Enterobacteriales; 29: g_Muribaculaceae_Ambiguous_taxa; 30: g_Christensenellaceae_R_7; 31: f_Pasteurellaceae; 32: o_Pasteurellales; 33: g_Rodentibacter; 34: g_Erysipelatoclostridium; 35: g_Eisenbergiella; 36: g_Flavonifractor; 37: g_Peptococcaceae_Uncultured; 38: f_Peptococcaceae; 39: f_Christensenellaceae; 40: f_Clostridiales_Family_XIII; 41: g_Eubacterium_nodatum; 42: f_Lachnospiraceae; 43: f_Prevotellaceae; 44: g_Prevotellaceae_NK3B31; 45: g_Lachnospiraceae_Uncultured; 46: g_Coprococcus_2; 47: g_Ruminococcaceae_UCG_014; 48: g_Ruminiclostridium_5; 49: p_Firmicutes; 50: f_Ruminococcaceae; 51: g_Lachnospiraceae_NA4A136; 52: g_Ruminococcus_1; 53: o_Gastranaerophilales; 54: c_Melainabacteria; 55: p_Cyanobacteria; 56: g_Oscillibacter; 57: g_Lachnospiraceae_ASF356; 58: g_Ruminococcaceae_UCG_005; 59: f_Ambiguous_taxa; 60: g_Ambiguous_taxa_2; and 61: g_Ruminiclostridium.

FIG. 9 shows the short chain fatty acid (SCFA) levels of rats in each group during puberty. *p-value<0.05, **p-value<0.01, ***p-value<0.001, ****p-value<0.0001.

FIG. 10 is a heatmap showing correlation between dominant taxa and SCFA levels. *p-value<0.05.

### DETAILED DESCRIPTION OF EMBODIMENTS

The description discloses some embodiments in such a detail that a person skilled in the art is able to utilize the embodiments based on the disclosure. Not all steps or features of the embodiments are discussed in detail, as many of the steps or features will be obvious to a person skilled in the art based on this disclosure.

It is further noted that, as used in this disclosure, the singular forms “a,” “an,” and “the” include plural referents unless expressly and unequivocally limited to one referent. The term “or” is used interchangeably with the term “and/or” unless the context clearly indicates otherwise.

As used herein, the term “about” generally means within 10%, 5%, 1%, or 0.5% of a given value or range. Alternatively, the term “about” means within an acceptable standard error of the mean when considered by one of ordinary skill in the art. Unless otherwise expressly specified, all of the numerical ranges, amounts, values, and percentages such as those for quantities of materials, durations of time periods, temperatures, operating conditions, ratios of amounts, and the likes disclosed herein should be understood as modified in all instances by the term “about.”

As used herein, the term “comprising” or “comprises” is used in reference to compositions, methods, and respective component(s) thereof, which are included in the present disclosure, yet open to the inclusion of unspecified elements.

As used herein, the term “treat,” “treating,” or “treatment” encompasses partially or completely preventing, ameliorating, mitigating, and/or managing a symptom, a disorder, or a condition associated with a disease. The term “treat,” “treating,” or “treatment” as used herein refers to application or administration of one or more therapeutic agent or surgery to a subject, who has a symptom, a disorder, or a condition associated with a disease, with the purpose to partially or completely alleviate, ameliorate, relieve, delay onset of, inhibit progression of, reduce severity of, and/or reduce incidence of one or more symptoms, disorders, or conditions associated with the disease. Treatment may be administered to a subject who exhibits only an early sign of such symptoms, disorders, and/or conditions for the purpose of decreasing the risk of developing the symptoms, disorders, and/or conditions associated with a disease.

As used herein, the term “preventing” or “prevention” refers to preventive or avoidance measures for a disease or symptoms or conditions of a disease, which include, but are not limited to, applying or administering one or more active agents to a subject who has not yet been diagnosed as a patient suffering from the disease or the symptoms or conditions of the disease but may be susceptible or prone to the disease. The purpose of the preventive measures is to avoid, prevent, or postpone the occurrence of the disease or the symptoms or conditions of the disease.

As used herein, the terms “patient,” “individual,” and “subject” are used interchangeably. The term “subject” means a human or animal. Examples of the subject include, but are not limited to, a rodent, a murine, a monkey, a guinea pig, a dog, a cat, a cow, a sheep, a pig, a horse, a rabbit, and a human. In some embodiments of the present disclosure, the subject is a mammal, e.g., a primate such as a human.

As used herein, the phrase “an effective amount” refers to the amount of an active ingredient (e.g., monoammonium glycyrrhizinate) that is required to confer a desired effect (e.g., inhibiting estrogen production) on the treated subject. Effective doses will vary, as recognized by one of ordinary skill in the art, depending on routes of administration, excipient usage, the possibility of co-usage with other treatment, and the condition to be treated.

As used herein, the term “administering” or “administration” refers to the placement of an active ingredient (e.g., monoammonium glycyrrhizinate) into a subject by a method or route which results in at least partial localization of the active ingredient at a desired site to produce a desired effect. The active ingredient described herein may be administered by any appropriate route known in the art including, but not limited to, oral or parenteral routes, including intraperitoneal, intravenous, intradermal, intramuscular, subcutaneous, or transdermal routes.

In at least one embodiment of the present disclosure, the composition may be administered to the subject orally, intravenously, subcutaneously, intradermally, intraperitoneally, intranasally, intramuscularly, topically, or through nebulization.

In at least one embodiment of the present disclosure, the composition may be administered to the subject 1 to 4 times per day, 1 to 7 times per week, or 1 to 30 times per month. In some embodiments, the composition is administered to the subject with a frequency selected from the group consisting of once per month, 2 times per month, 4 times per month, 6 times per month, 7 times per month, 8 times per month, 15 times per month, 20 times per month, once per week, 2 times per week, 3 times per week, 4 times per week, 5 times per week, 6 times per week, once every three days, 2 times every three days, 3 times every three days, once every two days, 2 times every two days, once per day, 2 times per day, and 3 times per day.

The composition of the present disclosure may be a pharmaceutical composition comprising a pharmaceutically acceptable excipient or an edible composition comprising a food excipient.

Depending on the type of the pharmaceutical compositions or the edible compositions, the pharmaceutically acceptable excipient and the food excipient may be independently chosen from any one or a combination of excipients known in the art. The choice of the pharmaceutically acceptable excipient and the food excipient depends partly upon the desired method of administration to be used.

As used herein, the term “pharmaceutically acceptable excipient” refers to a pharmaceutically acceptable material, composition, or vehicle, which does not abrogate the biological activity or properties of the active ingredient (e.g., glycyrrhizin), and is relatively non-toxic; that is, the material may be administered to an individual without causing an undesirable biological effect or interacting in a deleterious manner with any of the components of the composition in which it is contained.

As used herein, the term “food excipient” refers to an edible material that may have no bioactivity itself but improve the absorption of nutrients and active ingredients into the body; for example, the food excipient may protect nutrients and ingredients until they arrive at the location where they are intended to be useful.

In at least one embodiment of the present disclosure, the pharmaceutically acceptable excipient or the food excipient may be selected from the group consisting of a filler, a binder, a preservative, a humectant, a buffering agent, a pH adjuster, an antifoaming agent, a disintegrating agent, a lubricant, a suspending agent, a wetting agent, a solvent, a surfactant, an acid, a flavoring agent, and any combination thereof.

The examples of the pharmaceutically acceptable excipient and the food excipient include, but are not limited to, polyethylene glycol (PEG), alkylene glycol, dimethyl sulfoxide (DMSO), propylene glycol, sebacic acid, an alcohol, stearic acid, calcium stearate, sorbitan stearate, glyceryl monostearate, microcrystalline cellulose, silicon dioxide, gelatin, fat, glycerin, dietary fiber, alginate, pectin, carrageenan, amidated pectin, xanthan, gellan gum, karaya gum, rhamsan, welan, gum ghatti, gum arabic, cera alba, sorbitan palmitate, cetyl palmitate, cetyl 2-ethyl hexanoate, sorbitan oleate, phenoxyethanol, petrolatum, sodium chloride, methylparaben, propylparaben, cetearyl alcohol, lactose, sucrose, mannitol, fructose, sorbitol, xylitol, erythritol, trehalose, hydroxypropyl cellulose, hydroxyethyl cellulose, sodium benzoate, benzoic acid, benzyl alcohol, sorbic acid, citric acid, phosphoric acid, succinic acid, fumaric acid, tartaric acid, ascorbic acid, simethicone, dimethicone, and light anhydrous silicic acid.

In at least one embodiment of the present disclosure, the composition comprises the pharmaceutically acceptable excipient and/or the food excipient in an amount of from about 0.1% to about 60% (w/w), such as about 10% to about 50%, about 10% to about 30%, about 10% to about 20%, about 8% to about 15%, about 5% to about 50%, about 5% to about 30%, about 5% to about 20%, about 5% to about 15%, about 1% to about 15%, about 1% to about 10%, about 0.5% to about 15%, about 0.1% to about 10%, or about 0.1% to about 5% (w/w).

The composition of the present disclosure may comprise the glycyrrhizin or the salt or derivative thereof as a sole active ingredient for preventing or treating precocious puberty. In other words, the glycyrrhizin or the salt or derivative thereof serves as the only active ingredient for preventing or treating precocious puberty in the composition. In this embodiment, the present disclosure provides a safe and effective therapy for preventing or treating precocious puberty by the use of the glycyrrhizin or the salt or derivative thereof alone as the active ingredient.

Alternatively, in other embodiments, in addition to the glycyrrhizin or the salt or derivative thereof, the composition may be administered to a subject in combination with an additional therapy for preventing or treating precocious puberty unless the effect of the present disclosure is inhibited. In these embodiments, the glycyrrhizin or the salt or derivative thereof may be administered simultaneously or sequentially with the additional therapy. For example, the additional therapy is another active ingredient for preventing or treating precocious puberty, and the glycyrrhizin or the salt or derivative thereof is administered with the another active ingredient in a single formulation or in separate formulations.

In at least one embodiment of the present disclosure, the composition is in a form selected from the group consisting of an injection formulation, a dry powder, a tablet, an oral liquid, a flake, a film, a lozenge, a capsule, a granule, a pill, a gel, a lotion, an ointment, an emulsifier, a paste, a cream, and a salve.

### EXAMPLES

Exemplary embodiments of the present disclosure are further described in the following examples, which should not be construed to limit the scope of the present disclosure.

### Materials and Methods

The materials and methods used in the following Examples 1 to 5 are described in detail below. The materials used in the present disclosure but unannotated herein are commercially available.

### (1) Cohort Introduction

The Taiwan Puberty Longitudinal Study (TPLS) was a multicenter project involving several hospitals in Taiwan. A total of more than 2,000 female adolescents aged from 6 to 16 years and male adolescents aged from 9 to 17 years had been recruited from pediatric endocrine clinics since 2018. Anthropometric measurements, pubertal stages exams, and interviews about physical activity and sleep quality were conducted quarterly. A comprehensive evaluation, including 24-hour food recall, fasting blood test, and parental interviews about their children's physical activity and sleep quality, was performed annually. Participants were followed up until reaching the late pubertal stages.

### (2) Assessment of Glycyrrhizin and Added Sugar Intake

Thorough market search, a comprehensive and semiquantitative food frequency questionnaire (FFQ), was created for glycyrrhizin intake evaluation in advance. A total of 39 glycyrrhizin-containing food and beverage products were listed in this non-nutritive sweetener food frequency questionnaire (NNS-FFQ)^{[1]}. The questionnaire was used to screen the level of glycyrrhizin consumption. The interval scale for frequency was measured as never or a number of times per day or per week. Glycyrrhizin consumption was described as the proportion relative to acceptable daily intake (ADI) set by the Joint FAO/WHO Expert Committee on Food Additives[2]. The glycyrrhizin exposure dose in % ADI was finally categorized as tertiles, ranging from T1 (0 to 0.0006), T2 (0.0006 to 0.002) to T3 (>0.002). Concomitantly, the added sugar consumption of participants in TPLS cohort was evaluated to demonstrate its association with early puberty and compare it with that of glycyrrhizin. Most of the added sugar came from sugar-sweetened beverages (SSB). The added-sugar exposure dose in % ADI was also categorized as tertiles, ranging from T1 (0 to 0.0013), T2 (0.0013 to 0.0044) to T3 (>0.0044).

### (3) Definition of Outcomes

Precocious puberty (PP) was diagnosed in the presence of the following criteria: secondary sexual characteristics happen before the age of 8 years old, bone age advance greater than 2 years above chronological age, and peak luteinizing hormone (LH)≥5 after the gonadotropin-releasing hormone (GnRH) stimulation test^{[3]}.

### (4) Measurements of Confounders

Body mass index (BMI) was calculated as weight in kilograms divided by height in meters squared and was then transformed into z-scores (zBMI) based on age- and sex-related reference charts for Taiwanese children^{[4]}. Participants were asked for their habitual activities, and the amount of time they spend for specific physical activities. The intensity of each activity was described as metabolic equivalent of task (MET)^{[5]}. Daily metabolic equivalent value (METD) was calculated by summarizing mean of the standard MET of each activity multiplied by the average daily duration. The sleep quality scores, ranging from 0 to 21, were assessed by the Pittsburgh Sleep Quality Index (PSQI) through a self-reported questionnaire^{[6]}. The total energy intake daily in kcal was estimated by the summary of energy intake from three major macronutrients, including carbohydrates, fat, and proteins. Parental education was assessed by interviews and classified as “senior high school and below,” “college,” or “graduate school and higher.”

### (5) Microbiome Analysis

Children who had history of using antibiotics and probiotics within three months prior to fecal sample collection were excluded from the microbiome analysis. The bacterial DNA was extracted by Qiagen DNA Mini Kit and stored at −80° C. until using in further downstream steps including PCR assays and 16S RNA sequencing. The universal primers 341F (5′-CCTACGGGNGGCWGCAG-3′) (SEQ ID NO. 1) and 805R (5′-GACTACHVGGGTATCTAATCC-3′) (SEQ ID NO. 2) were used for PCR performed to amplify the hypervariable V3-V4 region of the bacterial 16S rRNA gene. High-throughput sequencing was then performed on an Illumina MiSeq 2000 sequencer with a MiSeq Reagent kit v3 (Illumina). After sequencing, the frequency table with relative abundance, taxonomy and phylogenetic tree was constructed. Groups of closely related individuals were considered as operational taxonomic units (OTUs). Alpha diversity was measured by calculating the Total Observation, Shannon and Simpson index. Beta diversity was evaluated using the principle coordinate analysis (PCoA) on Bray-Curtis dissimilarity matrix. The linear discriminant analysis effect size (LEfSE) was used to determine dominant taxa most likely to explain differences between groups.

### (6) Statistical Analysis for Human Study

Odds ratios (ORs) for precocious puberty in participants consumed different doses of glycyrrhizin and added sugar were calculated from generalized linear models with logistic regression using glm( ) function. Models were adjusted for gender, age, zBMI, exercise time, sleep quality, total energy intake, and parental education. Atwo-tailed p-value of <0.05 was considered statistically significant. All analyses were performed using R software (version 4.0.2; RStudio; R Foundation for Statistical Computing, Inc., Boston, MA, USA).

### (7) Animal Model

A total of 129 female neonate Sprague-Dawley rats were housed in a humidity (67±1.5%) and temperature (24±2° C.)-controlled housing room of the Taipei Medical University's Laboratory Animal Center with a 12-hour light/dark cycle. They had free access to food and water.

Such rats were divided into Control, Danazol, MAG (monoammonium glycyrrhizinate), Danazol+Low-MAG (100 μg/kg/day), Danazol+Medium-MAG (200 μg/kg/day), and Danazol+High-MAG (400 μg/kg/day) groups. The day at birth was termed postnatal day (PND). Litters belonging to danazol-induced PP groups were given a single subcutaneous injection of 300 μg danazol (Sigma-Aldrich) dissolved in 30 μL vehicle composed of propylene glycerol and ethanol (1:1)^{[7]}. MAG was administered using oral gavage since PND 13. Offspring were weaned onto a normal chow diet and water at PND 21. Body weight was measured daily. The rats were examined daily for the vaginal opening, which was considered as a marker of pubertal onset.

All experimental procedures involving the use of animals were conducted in accordance with the guidelines and approved by the Taipei Medical University's Institutional Animal Use and Care Committee (IACUC).

### (8) MAG Preparation

MAG was prepared freshly at the beginning of the experiment daily by mixing MAG powder with sterilized water. The volume fed each day was calculated based on the MAG solubility (20 mg/mL), maximum volume tolerated (10 mL/kg/time), intended dose (either 100, 200, or 400 μg/kg/day), and the rat's body weight on that day. Those who were not in the MAG-fed groups were fed with the same volume of sterilized water. MAG doses were chosen based on existing guidelines for safe dosage in humans and previous work in animals. In more detail, the doses were about 5 to 20 times lower than the no-observed-adverse-effect-levels (NOAELs)^{[8]}. They reflect the normal physiological intake of this sweetener.

### (9) Animal Experimental Protocols

### (9-1) Experiment 1

In this experiment, rats were fed with MAG daily until the end of experiment to maintain the long-term effect of drug tested. Vaginal smear was performed daily until they established three consecutive regular 4- or 5-day-cycles. The day of vaginal opening was the primary outcome. Moreover, the day of the setup of the regular cycle and the day of the establishment of 3 regular cycles were observed as secondary outcomes. The first day of diestrus or metestrus of the first cycle in the 3 consecutive regular estrous cycles was determined as the day of the setup of the regular cycle^{[7]}. The last day of diestrus of the last cycle in the 3 consecutive regular estrous cycles was determined as the day of establishment of 3 regular cycles^{[9]}.

### (9-2) Experiment 2

In this experiment, rats were raised until the model group (danazol) experienced vaginal opening. At that time, all of the rats were then sacrificed to (1) compare the differences between serum estrogen and luteinizing hormone (LH) levels; (2) compare the mRNA expression of GnRHa, k, Lin28a, Lin28b, let7a, let7b, GPR54, netrin-1, UNC5C, and MKRN3 in the hypothalamus using qPCR; and (3) compare the number of GnRH and kisspeptin neurons in the hypothalamus using immunohistochemistry (IHC).

Overnight fasting blood samples for glucose measurement were collected from tail veins two days before sacrifice. Blood samples for LH, estrogen, and other routine laboratory tests were collected from the abdominal aorta when sacrificing. Insulin resistance was then estimated using the triglyceride glucose (TyG) index. These results were needed to confirm the safety of long-term MAG. Also, the fecal samples were collected during these days to analyze gut microbiome, short chain fatty acids (SCFA), and reserve material for fecal microbiota transplantation (FMT) in Experiment 3.

On the days of the vaginal opening in the model group, all animals were exsanguinated with normal saline following anesthesia. After perfusion, the hypothalamus was retrieved. Half of them were fixed in the 10% neutral-buffered formalin for IHC experiments, while the remaining half was embedded in RNAprotect reagent for qPCR experiments later. Uterus and bilateral ovaries were dissected out from surrounding fat to measure the net weight and coefficient weight (weight of reproductive organs relative to body weight, as mg/100 mg body weight). The intestinal mucosa was harvested and put in normal saline to investigate tight junction proteins later.

### (9-3) Experiment 3

In this experiment, the fecal material collected in Experiment 2 (from donor) was transplanted to mice (recipient) raised in a specific-pathogen free environment. Pathogen free mice model was established by feeding 25 mL of cocktail antibiotics, which contain 50 mg of ampicillin, 50 mg of neomycin, 50 mg of metronidazole, and 25 mg of vancomycin.

### (10) Biochemical Analysis

Blood glucose in rodents was measured using the BioSystem glucose GOD/Peroxidase kit. LH levels were measured using the Enzo LH ELISA kit following the manufacturer's instructions. Samples for estrogen and other routine test were sent to a clinical laboratory.

### (11) Statistical Analysis for Animal Study

Data were presented as means±standard deviation. Statistical analysis was performed on raw data using one-way analysis of variance (ANOVA). Comparisons among groups were made using the post hoc Tukey-HSD test. Day of vaginal opening, day of setup of regular cycle and day of establishment of 3 regular cycles were analyzed using survival analysis and were compared using the log-rank test. A two-tailed p-value<0.05 was considered significant.

### Example 1: Association Between Glycyrrhizin Consumption and Risk of Precocious Puberty

To assess the association of glycyrrhizin consumption and precocious puberty from a prospective human cohort, a total of 1,014 subjects were recruited. The demographic characteristics of participants stratified by gender were shown in Table 1 below.

TABLE 1
Baseline characteristics of study population

| Characteristics | Female | | Male | | P |
|---|---|---|---|---|---|
| Number of | 701 | (69.13) | 313 | (30.87) | <0.001 |
| participants, n (%) | | | | | |
| Age (years) | 9.77 ± 0.26 | | 11.79 ± 1.73 | | <0.001 |
| Weight (kg) | 34.22 ± 0.92 | | 42.27 ± 12.72 | | <0.001 |
| Body mass index (kg/m²) | 17.82 ± 0.47 | | 19.31 ± 4.00 | | <0.001 |
| Sleep quality score | 4.43 ± 0.14 | | 4.12 ± 2.84 | | 0.533 |
| Total calorie intake | 1532.82 ± 40.48 | | 1679.50 ± 314.21 | | <0.001 |
| (kcal/day) | | | | | |
| Parental education, n (%) | | | | | 0.043 |
| Senior high school | 127 | (18.12) | 57 | (18.21) | |
| and below | | | | | |
| College | 264 | (37.66) | 91 | (29.07) | |
| Graduate school | 310 | (44.22) | 165 | (52.72) | |
| and higher | | | | | |
| Physical activity | | | | | 0.408 |
| METD, n (%) | | | | | |
| Mild (<3) | 26 | (3.71) | 3 | (0.96) | |
| Moderate (3-6) | 191 | (27.25) | 61 | (19.49) | |
| Vigorous (>6) | 484 | (69.04) | 249 | (79.55) | |
| Precocious | 369 | (52.64) | 99 | (31.63) | <0.001 |

puberty, n (%)

METD: daily metabolic equivalent value

It was found that average age, body weight, BMI, and total energy intake were higher in boys than in girls (p<0.001). There was no difference in sleep quality scores and physical activity levels between two genders. There were 468 subjects with PP. The proportion of PP patients was significantly higher in girls than in boys (p K 0.001).

After fully adjustment, glycyrrhizin consumption was shown to reduce the risk of PP, whereas added sugar was associated with increased PP. The benefit only occurred with a certain dose of glycyrrhizin, whereas higher levels of intake no longer protected participants from PP (Table 2). This dose-dependent effect was driven by the differences in female.

TABLE 2
Sweeteners intake and risk of precocious puberty

| | Exposure | Total | | | | Female | | | | Male | | | |
|---|---|---|---|---|---|---|---|---|---|---|---|---|---|
| Sweeteners | dose* | OR | 95% CI | | P | OR | 95% CI | | P | OR | 95% CI | | P |
| Glycyrrhizin | 0 | Ref | — | — | — | Ref | — | — | — | Ref | — | — | — |
| | T1 | 0.607 | 0.388 | 0.949 | 0.029 | 0.479 | 0.284 | 0.810 | 0.006 | 1.384 | 0.591 | 3.237 | 0.454 |
| | T2 | 0.687 | 0.441 | 1.069 | 0.096 | 0.826 | 0.496 | 1.376 | 0.463 | 0.422 | 0.146 | 1.218 | 0.111 |
| | T3 | 0.857 | 0.545 | 1.337 | 0.490 | 0.969 | 0.574 | 1.636 | 0.905 | 0.656 | 0.246 | 1.749 | 0.399 |
| Added sugar | 0 | Ref | — | — | — | Ref | — | — | — | Ref | — | — | — |
| | T1 | 1.130 | 0.785 | 1.627 | 0.509 | 1.063 | 0.690 | 1.539 | 0.782 | 1.085 | 0.552 | 2.129 | 0.814 |
| | T2 | 1.651 | 1.148 | 2.374 | 0.007 | 1.907 | 1.230 | 2.955 | 0.004 | 1.029 | 0.523 | 2.025 | 0.834 |
| | T3 | 1.359 | 0.943 | 1.957 | 0.100 | 1.775 | 1.149 | 2.743 | 0.010 | 0.544 | 0.254 | 1.164 | 0.117 |

*Exposure dose was estimated as the proportion of daily intake to acceptable daily intake (ADI) and was categorized as tertiles, referencing the no intake group. Models were adjusted for gender, age, zBMI, exercise time, sleep quality, total energy intake, and parental education.
Ref: reference; CI: confidence interval; OR: odds ratio.

### Example 2: Association Between Glycyrrhizin Consumption and Gut Microbiome

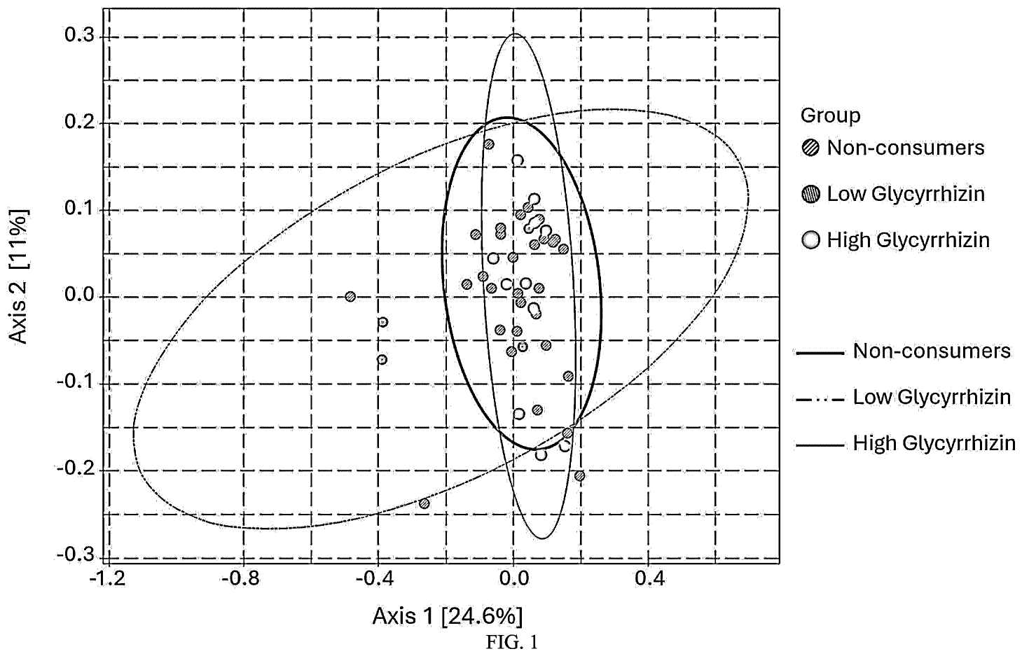

Based on the human study as descried in Example 1, it was found that beta diversity of low-glycyrrhizin users and non-consumers were significantly distinguishable (p=0.048) (FIG. 1).

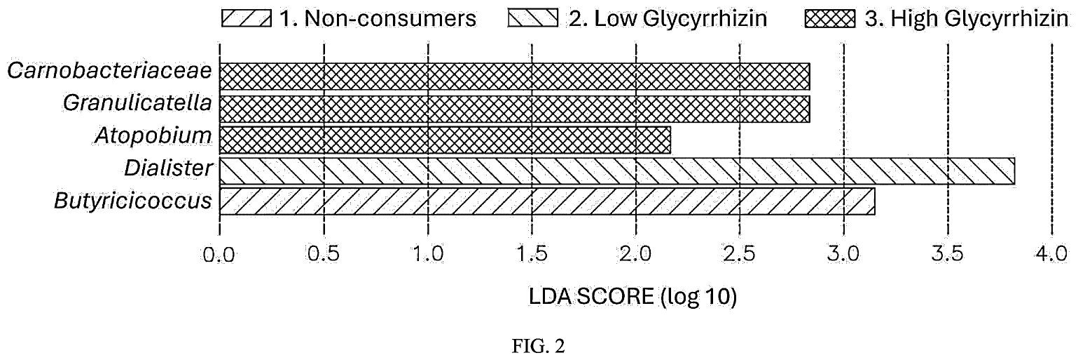

Further, as shown in FIG. 2, the dominant taxa were identified in each group, including genera Butyricicoccus in Non-consumers group, Dialister in Low-Glycyrrhizin group, and Atopobium and Granulicatella and family Carnobacteriaceae in High-Glycyrrhizin group.

### Example 3: Effects of Glycyrrhizin on Estrous Cycle

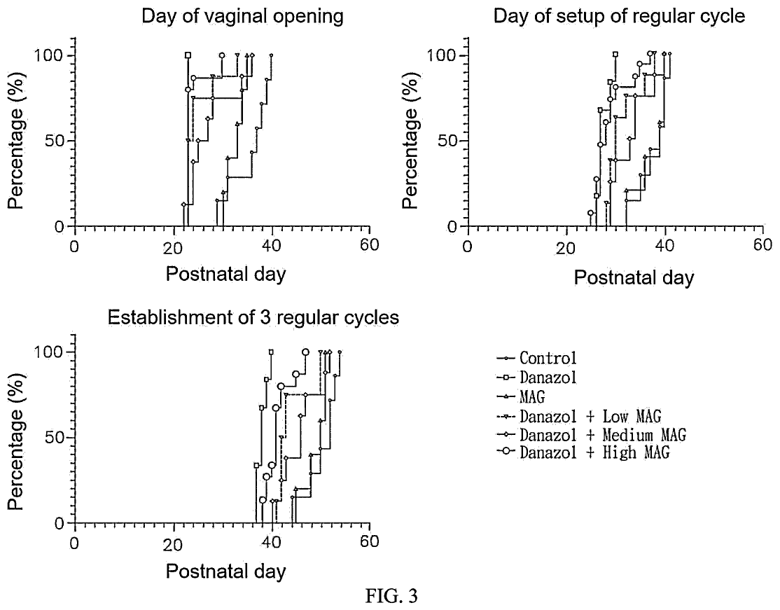

In animal Experiment 1, the direction of effect of glycyrrhizin was established by observing the whole estrous cycle. It was found that MAG protected rats against PP following a dose-dependent manner, in which the most obvious effects were observed with Low- and Medium-MAG groups (Table 3 and FIG. 3).

TABLE 3
Different puberty-related time points of the rats

| | | | | Danazol + | Danazol + | Danazol + |
|---|---|---|---|---|---|---|
| | Control | Danazol | MAG | Low-MAG | Med-MAG | High-MAG |
| | (n = 7) | (n = 6) | (n = 5) | (n = 8) | (n = 8) | (n = 15) |
| Day of vaginal | 35.71 ± 4.15* | 23.00 ± 0.00 | 32.60 ± 2.07* | 25.13 ± 3.60* | 27.50 ± 5.01* | 24.00 ± 2.45 |
| opening | | | | | | |
| Day of setup | 37.71 ± 3.25* | 27.67 ± 1.51 | 37.40 ± 3.44* | 31.50 ± 3.63* | 33.38 ± 4.07* | 28.93 ± 3.62 |
| of regular | | | | | | |
| cycle | | | | | | |
| Day of | 50.43 ± 3.46* | 38.17 ± 1.17 | 49.00 ± 2.55* | 44.13 ± 3.68* | 45.88 ± 4.19* | 41.47 ± 2.85* |
| establishment | | | | | | |
| of 3 regular | | | | | | |
| cycles | | | | | | |

*p < 0.05 versus Danazol group using log-rank test.
MAG: monoammonium glycyrrhizinate

There was no difference in body weight during puberty among groups (p=0.448). Uterine and ovarian coefficient weights were similar between groups (p=0.566 and 0.850, respectively). There were no safety concerns regarding liver enzyme, renal function, albumin, electrolyte panel, and lipid profiles (all p>0.05). Insulin resistance at the end of the experiment, mirrored by the TyG index, did not differ between groups, with the exception of a higher value in High-MAG than that of Medium-MAG (p=0.048).

### Example 4: Mechanistic Pathway of Glycyrrhizin Via the Gut Microbiome

In animal Experiment 2, the mechanistic pathway of glycyrrhizin was estimated via gut microbiome analysis, quantitative polymerase chain reaction (qPCR), and immunohistochemistry (IHC).

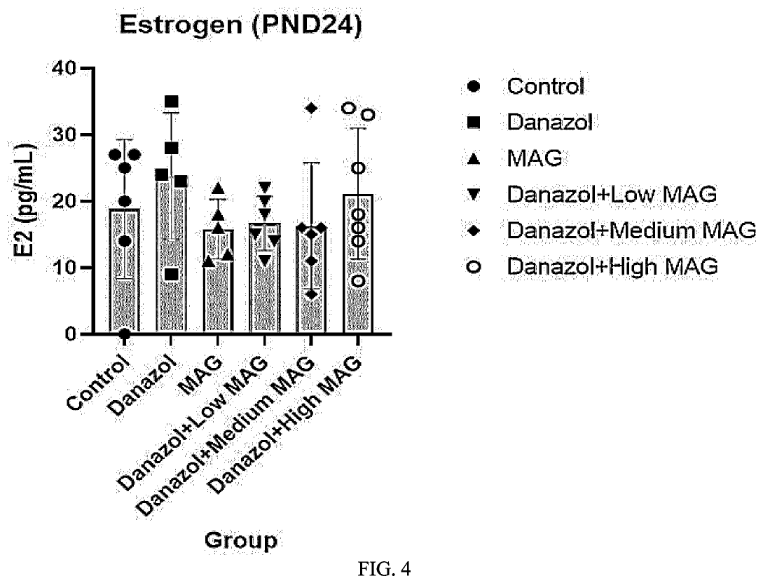

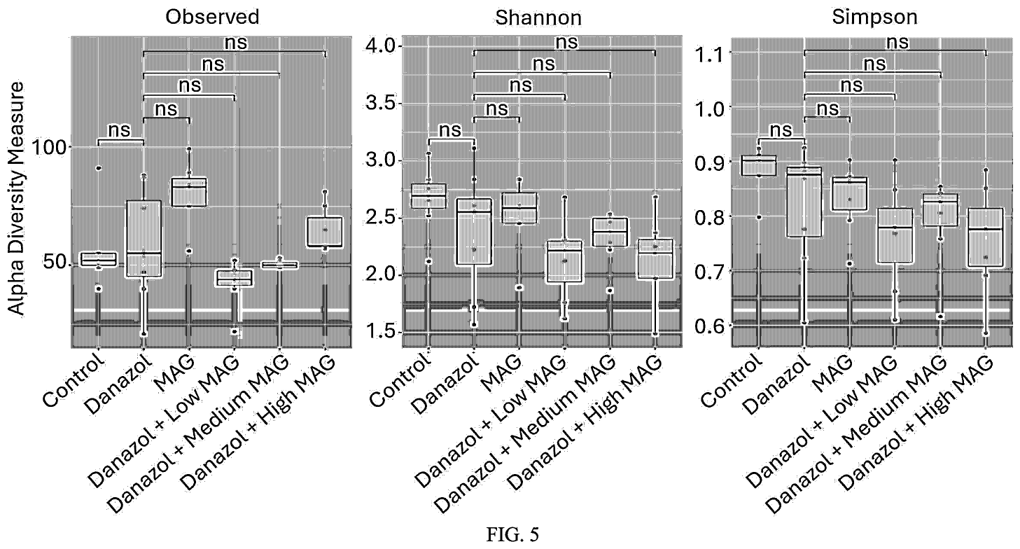

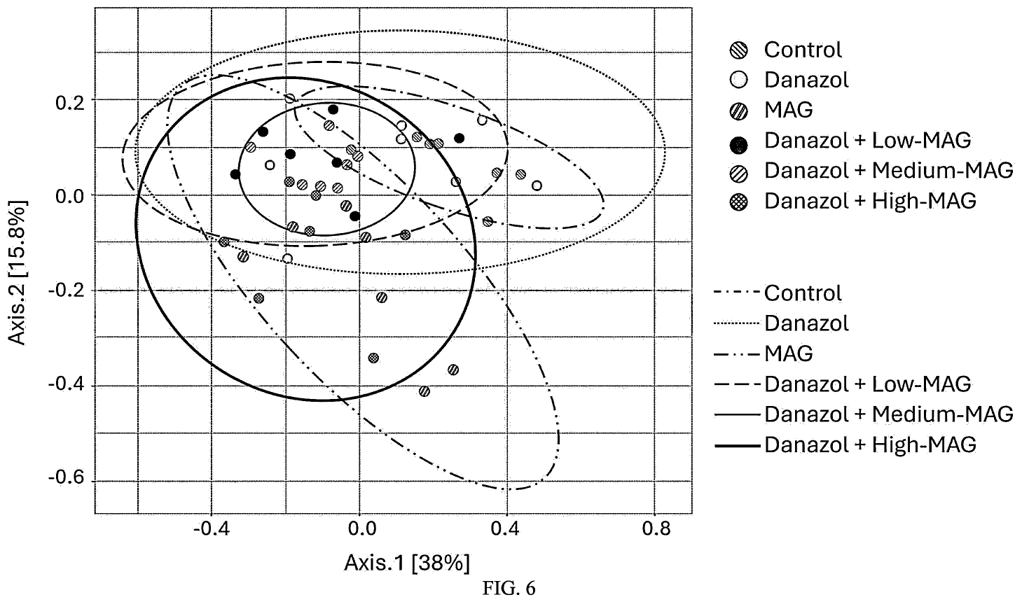

It was found that the day of vaginal opening results was consistent with serum estrogen level, which demonstrated a trend towards higher values in Danazol and High-MAG groups (FIG. 4). Further, as shown in FIG. 5, alpha diversity was comparable among groups. Meanwhile, beta diversity of Danazol group was significantly different from those of Low- and High-MAG groups (FIG. 6).

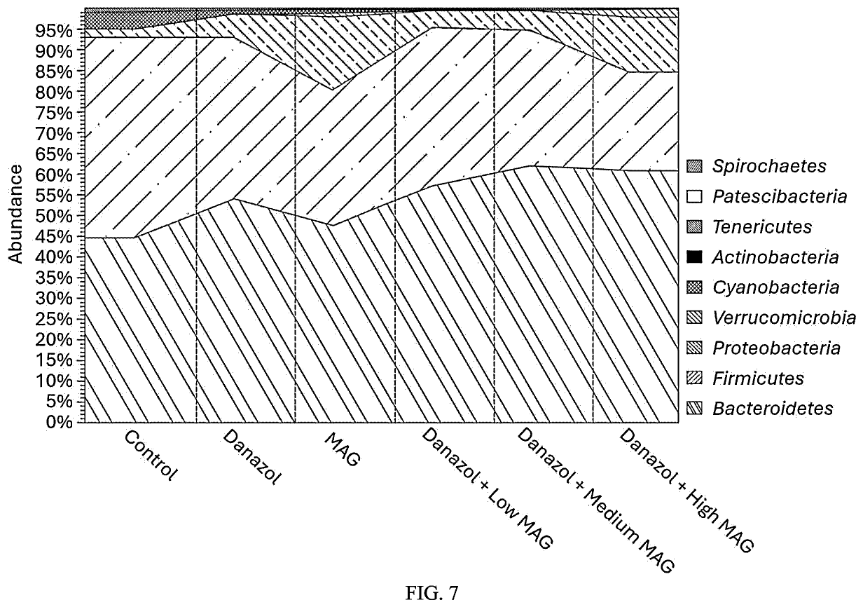

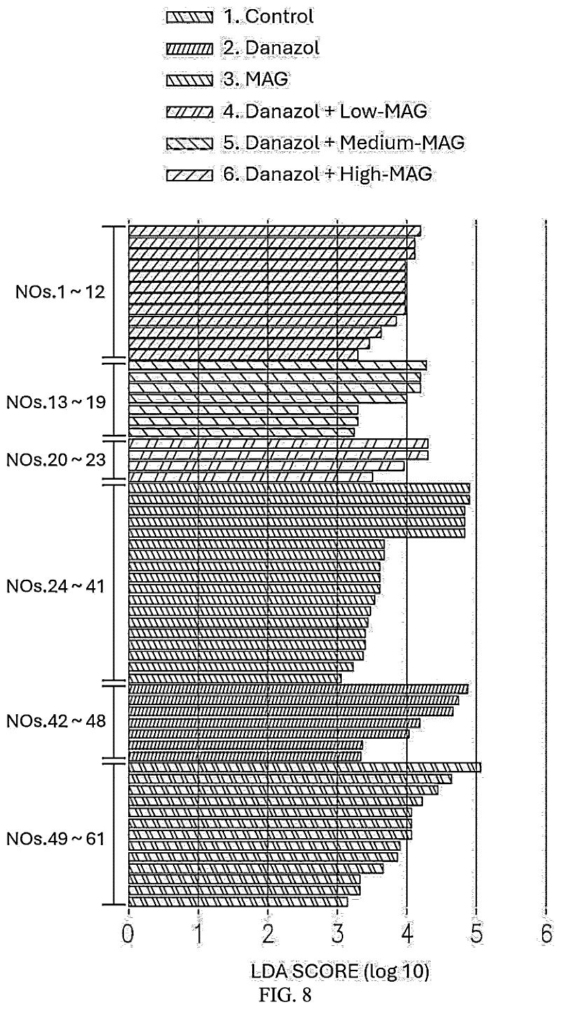

The use of MAG resulted in a decreased shift in Firmicutes to Bacteroidetes ratio. This effect was dose-dependent (FIG. 7). Several dominant taxa could be identified using LEfSE analysis (FIG. 8).

Enriched taxa of Danazol group as shown in FIG. 8, including Lachnospirace, Prevotellaceae, Coprococcus and Ruminococcaceae, were previously found to increase in central precocious puberty (CPP) patients^{[10, 11]}. In contrast, those enriched in MAG-fed rats, including Romboutsia and Allobaculum, were found to be negatively correlated with leptin or LH levels^{[11, 12]}.

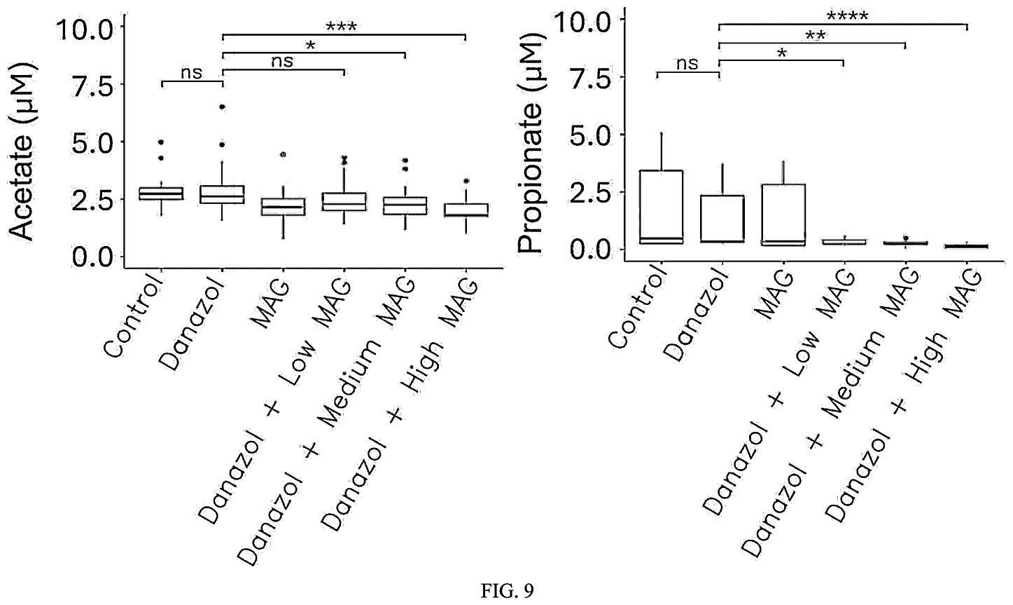

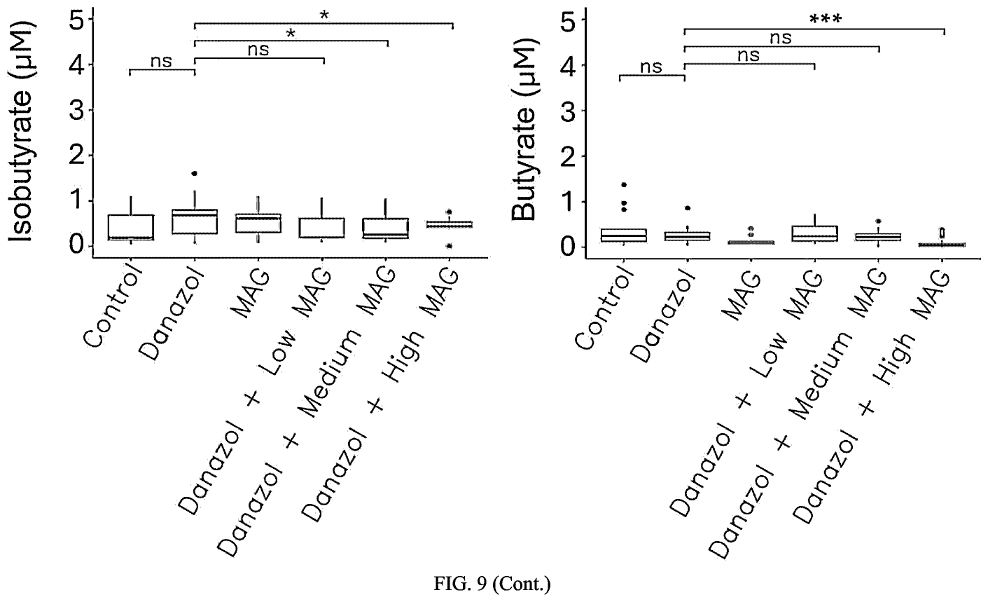

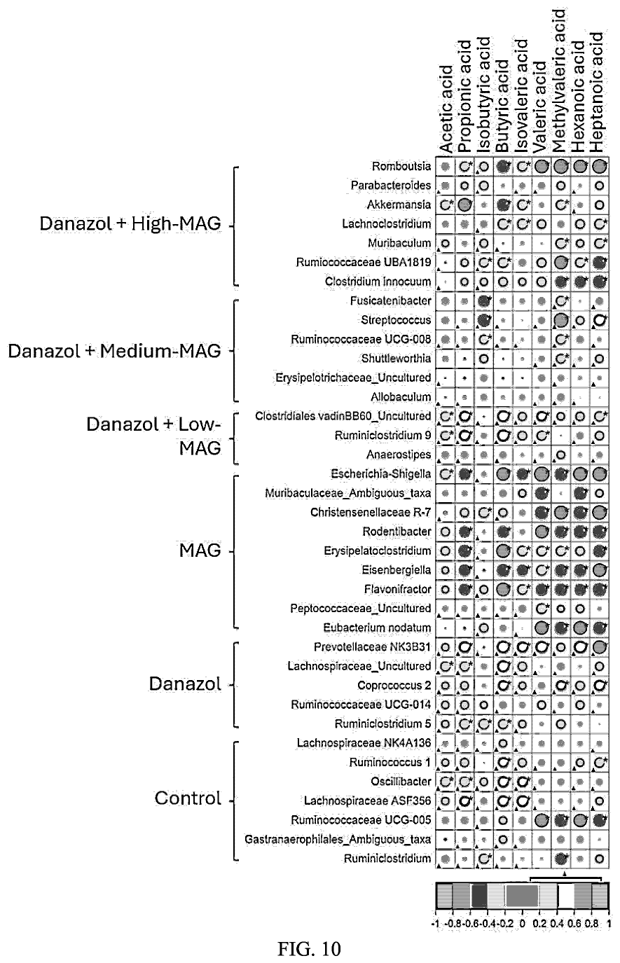

SCFA levels during puberty were decreased in accordance with increased dose of MAG (FIG. 9). Heatmap demonstrated that many taxa in MAG-fed rats were negatively correlated with SCFA levels, particularly in Medium- and High-MAG groups (FIG. 10).

### Example 5: Fecal Microbial Transplant Study

In animal Experiment 3, the fecal microbiota transplantation (FMT) was performed to further confirm that the effect of MAG was mediated by the gut microbiome. It was found that transplant of previously fed-MAG feces sample successfully recovered the Danazol-induced early vaginal opening (Table 4).

TABLE 4
Effect of FMT on days of vaginal opening

| | | | Danazol + | Danazol + |
|---|---|---|---|---|
| | Control | Danazol | 1-FMT | 2-FMT |
| Group | (n = 5) | (n = 6) | (n = 8) | (n = 8) |
| Day of | 30.80 ± | 24.33 ± | 26.25 ± | 28.25 ± |
| vaginal | 1.36^{#} | 0.33* | 1.46* | 0.88*^{#} |
| opening | | | | |
| (PND) | | | | |

*Significant different compared to Control group
^{#}Significant different compared to Danazol group
2-FMT: double dosage as compared with 1-FMT

The embodiments described hereinbefore may be used in any combination with each other. Several of the embodiments may be combined together to form a further embodiment. A compound or a method, disclosed herein, may comprise at least one of the embodiments described hereinbefore. It will be understood that the benefits and advantages described above may relate to one embodiment or may relate to several embodiments. The embodiments are not limited to those that solve any or all of the stated problems or those that have any or all of the stated benefits and advantages.

### REFERENCE

[1] Chu Y Y, Chen Y H, Hsieh R H, et al. Development and Validation of the Chinese Version Non-Nutritive Sweetener Food Frequency Questionnaire with Urinary Biomarker in Children and Adolescents. Public Health Nutr. 2022:1-23.
[2] World Health Organization (WHO). Evaluations of the Joint FAO/WHO Expert Committee on Food Additives (JECFA). Accessed May 20, 2022.
[3] Latronico A C, Brito V N, Carel J C. Causes, diagnosis, and treatment of central precocious puberty. Lancet Diabetes Endocrinol. 2016; 4(3):265-274.
[4] Chen W, Chang M H. New growth charts for Taiwanese children and adolescents based on World Health Organization standards and health-related physical fitness. Pediatr Neonatol. 2010; 51(2):69-79.
[5] Haskell W L, Lee I M, Pate R R, et al. Physical activity and public health: updated recommendation for adults from the American College of Sports Medicine and the American Heart Association. Med. Sci. Sports Exerc. 2007; 39(8):1423-1434.
[6] Buysse D J, Reynolds C F, 3rd, Monk T H, et al. The Pittsburgh Sleep Quality Index: a new instrument for psychiatric practice and research. Psychiatry Res. 1989; 28(2):193-213.
[7] Tian Z, Zhao H, Sun Y, et al. Evaluation of the true precocious puberty rats induced by neonatal administration of Danazol: therapeutic effects of nourishing “Yin”-Removing “Fire” Chinese herb mixture. Reprod. Biol. Endocrinol. 2005; 3:38.
[8] Bijlsma J. A., Van Vloten P., Van Gelderen C. E. M., et al. Onderzoek naar de effecten van verschillende doseringen glycyrrhizine bij gezonde vrouwelijke vrijwilligers [Study into the effects of different dosages of glycyrrhizin in health female volunteers; in Dutch]. RIVM Report. 1996 (348801004).
[9] Sun Y, Perry G N, Yu J, et al. Effect of nourishing “Yin”-removing “Fire” Chinese herbal mixture on hypothalamic kisspeptin expression in female precocious rats. J. Ethnopharmacol. 2010; 127(2):274-279.
[10] Huang C, Liu H, Yang W, et al. Distinct Gut Microbiota Structure and Function of Children With Idiopathic Central and Peripheral Precocious Puberty. Research Square; 2021.
[11] Dong G, Zhang J, Yang Z, et al. The Association of Gut Microbiota With Idiopathic Central Precocious Puberty in Girls. Front Endocrinol (Lausanne). 2019; 10:941.
[12] Ravussin Y, Koren O, Spor A, et al. Responses of gut microbiota to diet composition and weight loss in lean and obese mice. Obesity (Silver Spring). 2012; 20(4):738-747.

What is claimed is:

1. A method for preventing or treating precocious puberty in a subject in need thereof, comprising administering an effective amount of a composition to the subject, wherein the composition comprises glycyrrhizin or a salt or derivative thereof and at least one of a pharmaceutically acceptable excipient thereof and a food excipient thereof.

2. The method according to claim 1, wherein the salt or derivative of the glycyrrhizin is selected from the group consisting of glycyrrhizic acid, glycyrrhetic acid, monoammonium glycyrrhizinate, dipotassium glycyrrhizinate, sodium glycyrrhizinate, and calcium glycyrrhizinate.

3. The method according to claim 1, wherein the precocious puberty is central precocious puberty or peripheral precocious puberty.

4. The method according to claim 1, wherein after the administration, the composition inhibits early ovary growth, inhibits estrogen production, inhibits short chain fatty acids production, and/or increases body length in the subject.

5. The method according to claim 1, wherein after the administration, the composition increases a level of at least one of Dialister, Atopobium, Granulicatella, family Carnobacteriaceae, Romboutsia, and Allobaculum in the subject.

6. The method according to claim 1, wherein after the administration, the composition decreases a level of at least one of Lachnospirace, Prevotellaceae, Coprococcus, and Ruminococcaceae in the subject.

7. The method according to claim 1, wherein the glycyrrhizin or the salt or derivative thereof serves as a sole active ingredient for preventing or treating the precocious puberty in the composition.

8. The method according to claim 1, wherein the glycyrrhizin or the salt or derivative thereof is administered to the subject in an effective amount of from about 0.1 mg/day to about 2,000 mg/day.

9. The method according to claim 8, wherein the glycyrrhizin or the salt or derivative thereof is administered to the subject in an effective amount of from about 0.5 mg/day to about 1,000 mg/day.

10. The method according to claim 1, wherein the subject is of age from 2 to 18 years old.

11. The method according to claim 1, wherein the composition is administered to the subject in a period ranging from 1 month to 10 years.

12. The method according to claim 1, wherein the composition is administered to the subject orally, intravenously, subcutaneously, intradermally, intraperitoneally, intranasally, intramuscularly, topically, or through nebulization.

13. The method according to claim 1, wherein the composition is administered to the subject with a frequency selected from the group consisting of once per month, 2 times per month, 4 times per month, 6 times per month, 7 times per month, 8 times per month, 15 times per month, 20 times per month, once per week, 2 times per week, 3 times per week, 4 times per week, 5 times per week, 6 times per week, once every three days, 2 times every three days, 3 times every three days, once every two days, 2 times every two days, once per day, 2 times per day, and 3 times per day.

14. The method according to claim 1, wherein the glycyrrhizin or the salt or derivative thereof is in an amount of from about 0.001% to about 95% by weight in the composition.

15. The method according to claim 14, wherein the glycyrrhizin or the salt or derivative thereof is in an amount of from about 0.01% to about 80% by weight in the composition.

16. The method according to claim 1, wherein the composition is a pharmaceutical composition comprising the glycyrrhizin or the salt or derivative thereof and the pharmaceutically acceptable excipient thereof.

17. The method according to claim 1, wherein the composition is an edible composition comprising the glycyrrhizin or the salt or derivative thereof and the food excipient thereof.

18. The method according to claim 1, wherein the pharmaceutically acceptable excipient or the food excipient is selected from the group consisting of a filler, a binder, a preservative, a humectant, a buffering agent, a pH adjuster, an antifoaming agent, a disintegrating agent, a lubricant, a suspending agent, a wetting agent, a solvent, a surfactant, an acid, a flavoring agent, and any combination thereof.

19. The method according to claim 1, wherein the pharmaceutically acceptable excipient or the food excipient is selected from the group consisting of polyethylene glycol (PEG), alkylene glycol, dimethyl sulfoxide (DMSO), propylene glycol, sebacic acid, an alcohol, stearic acid, calcium stearate, sorbitan stearate, glyceryl monostearate, microcrystalline cellulose, silicon dioxide, gelatin, fat, glycerin, dietary fiber, alginate, pectin, carrageenan, amidated pectin, xanthan, gellan gum, karaya gum, rhamsan, welan, gum ghatti, gum arabic, cera alba, sorbitan palmitate, cetyl palmitate, cetyl 2-ethyl hexanoate, sorbitan oleate, phenoxyethanol, petrolatum, sodium chloride, methylparaben, propylparaben, cetearyl alcohol, lactose, sucrose, mannitol, fructose, sorbitol, xylitol, erythritol, trehalose, hydroxypropyl cellulose, hydroxyethyl cellulose, sodium benzoate, benzoic acid, benzyl alcohol, sorbic acid, citric acid, phosphoric acid, succinic acid, fumaric acid, tartaric acid, ascorbic acid, simethicone, dimethicone, and light anhydrous silicic acid.

20. The method according to claim 1, wherein the composition is in a form selected from the group consisting of an injection formulation, a dry powder, a tablet, an oral liquid, a flake, a film, a lozenge, a capsule, a granule, a pill, a gel, a lotion, an ointment, an emulsifier, a paste, a cream, and a salve.


SEQUENCE LISTING (2 sequences)

SEQ ID NO: 1 - DNA, 17 bp, synthetic construct
  source 1..17: mol_type = other DNA
cctacgggnggcwgcag

SEQ ID NO: 2 - DNA, 21 bp, synthetic construct
  source 1..21: mol_type = other DNA
gactachvgggtatctaatcc